(19) United States
(12) Patent Application Publication
(10) Pub. No.: US 2008/0169220 A1
(43) Pub. Date: Jul. 17, 2008

(54) Instant and continuous fragrance dispensing assembly, method of packaging, and method of using same

(75) Inventor: Kelly Gaines, Sugar Land, TX (US)

Correspondence Address: ANDREWS & KURTH, L.L.P., 600 TRAVIS, SUITE 4200, HOUSTON, TX, 77002, US

(73) Assignee: Gaines Group, LLC

(21) Appl. No.: 11/818,412

(22) Filed: Jun. 14, 2007

Related U.S. Application Data
(60) Provisional application No. 60/884,977, filed on Jan. 15, 2007.

(51) Int. Cl. B65D 69/00; A61L 9/00

(52) U.S. Cl. 206/576; 206/229; 206/223; 422/5

(57) ABSTRACT

A packaged kit for storing an instant fragrance dispensing assembly includes a storage container having a sealed interior chamber containing porous reeds in a fragranced liquid. The porous reeds and the fragranced liquid are put in the storage container, placed in contact with each other, and the storage container is sealed. The packaged kit provides instant and continuous fragrance to a room by opening the sealed storage container and transferring the porous reeds and the fragranced liquid from the storage container to a holding container. A portion of the porous reeds extend from the holding container and release the fragrance from the exposed fragrant liquid into the surrounding area.

### CROSS-REFERENCE TO RELATED APPLICATIONS

This application claims priority from U.S. Provisional Application Ser. No. 60/884,977, filed Jan. 15, 2007. Applicant incorporates by reference herein U.S. Provisional Application Ser. No. 60/884,977 in its entirety.

### BACKGROUND OF THE INVENTION

1. Field of the Invention

The present invention relates to fragrance dispensing assemblies, and more particularly relates to devices and techniques for dispensing fragrance instantly and continuously into a surrounding area and a method of packaging same.

2. Description of the Related Art

Various techniques exist for releasing fragrance into the air. These techniques are often used in homes, offices, or other enclosed areas in order to eliminate unpleasant odors or simply to add a pleasing fragrance into the air. Burning scented candles, for example, release fragrance from the melted wax but can be hazardous if left unattended. Furthermore, office buildings and other public places prohibit burning candles due to safety and/or fire code regulations.

Another technique used to emit a fragrant scent in the air is with the use of potpourri. Traditional potpourri comprises various mixtures of aromatic herbs, dried flowers, and spices blended with essential oils. The potpourri is usually contained within a bowl or a basket so that it can release its fragrance into the air. Typically, the potpourri scent is emitted in a very confined space and lasts only a short period of time. Within a day or two, the potpourri mixture is dried up and ready to be discarded. Fragrant oils can be sprayed or dripped onto the potpourri to enhance the aroma, but again the area in which this is effective is very small.

Another technique is to use the potpourri with an external heat source and water to cause the fragrance to become airborne through the vaporization process of boiling the water. The potpourri mixture is typically placed in a pot of water which is then heated. Although this technique is effective in making the aroma stronger and more widely spread, it has disadvantages. The disadvantages are that the heat source is typically either an electrical heating element or an open flame (such as a burning candle), both of which can be dangerous. Additionally, if the heated water is touched or spilled, it could burn badly, and, if all of the water in the pot is vaporized and the heat source remains on, the potpourri material itself can be set on fire.

Still another technique has been developed and used the last few years. This technique involves the use of slender reeds, typically made of wood, and fragranced liquid or oil placed in an open container or bottle. With the fragranced liquid in the container, one end of the reeds are inserted through the opening of the container into the fragranced liquid and a second end of the reeds extend above the container opening. The reeds each have a length such that when the first end comes to rest inside the container, the second end extends into the space above the container opening. The first end of the reeds are in the fragranced liquid in the container. The hygroscopic nature of the reed material allows the reeds to absorb the fragranced liquid in the hollow space between the walls of the wood cells. After a period of time, the fragranced liquid is drawn or absorbed to the upper ends of the reeds resulting in fragranced liquid evaporating and being diffused into the surrounding area.

The fragrant reed technique has the advantages of no open flame or heat source required to scent the air, it scents the air continuously and can be left unattended. However, this technique has the disadvantage of requiring a lengthy period of time before it begins to diffuse the scent into the room. This is due to the amount of time required for the reeds to absorb the fragranced liquid throughout the length of the reeds. For example, various commercially available fragrant reed products indicate that, for reeds having a length of approximately 10″, the reeds will draw and diffuse the fragrance within approximately 36 to 48 hours. The fragrant reeds will then continue to diffuse the fragrance throughout the room for extended periods of time depending on the amount of fragranced liquid and number of reeds being used. For example, 10 reeds and 6 ounces of fragranced liquid will typically continue to scent an area for several weeks.

It would be desirable to provide a fragrance dispensing assembly and method which requires no open flame or heat source. It would also be desirable to provide a fragrance dispensing assembly which scents the air continuously and can be left unattended. Additionally, it would be desirable to provide a fragrance dispensing assembly and technique that dispenses a fragrance throughout a room immediately without the extended waiting period currently required with commercially available fragrant reeds.

### SUMMARY OF THE INVENTION

The preferred embodiment of the present invention is a technique and assembly for providing a fragrance dispensing device require no open flame or heat source and which scents a room immediately and continuously and can be left unattended.

### BRIEF DESCRIPTION OF THE DRAWINGS

A better understanding of the present invention can be obtained when the following detailed description of preferred embodiments is considered in conjunction with the drawings in which:

FIG. 1 is an elevation view of preferred embodiment of the present invention having a plurality of reeds and fragranced liquid packaged in a storage container, the storage container illustrated in an open condition with a removable cap positioned above the storage container;

FIG. 2 is a perspective view of the fragranced liquid and plurality of reeds being transferred from the storage container into a holding container;

FIG. 3 is a perspective view of the fragranced reeds positioned in the holding container containing the fragranced liquid;

FIG. 4 is another view similar to FIG. 1 showing an alternative embodiment of the present invention in the packaged condition; and

FIGS. 5 and 6 show alternative embodiments of the storage container according to the present invention.

### DETAILED DESCRIPTION OF PREFERRED EMBODIMENTS

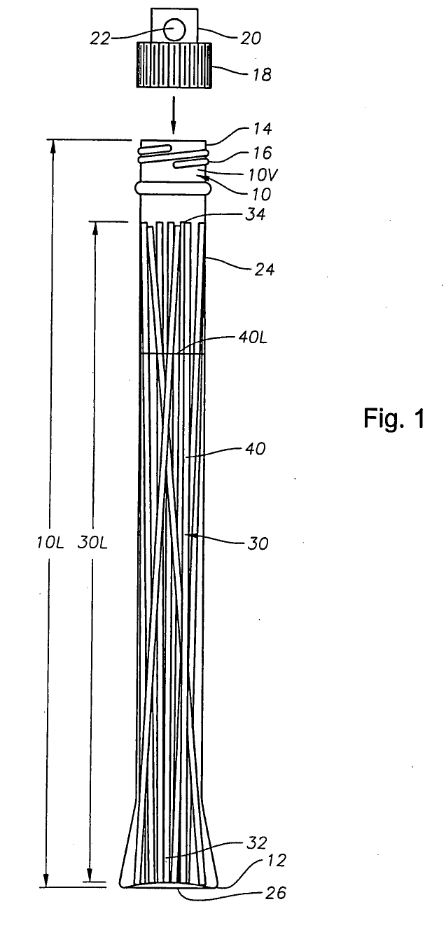

With reference to the drawings, the preferred embodiments of the present invention will now be described in detail. Referring to FIG. 1, a storage container 10 is shown having a first end 12 and a second end 14 defining a length 10L therebetween. Preferably, the storage container 10 includes a wall 24 that is generally circular in cross-section along its length 10L. The wall 24 is joined to a base member 26 at the first end 12. It is to be understood that the joinder of the container wall 24 to the base member 26 is fluid-tight. As shown in FIG. 1, the first end 12 is preferably larger in diameter than the diameter along the remaining length of the storage container 10 to provide stability if the storage container 10 is stood upright on the base member 26.

In the embodiment shown in FIG. 1, the second end 14 of the storage container 10 is provided with a threaded portion 16 adapted to correspondingly engage a threaded cap 18. It is to be understood that the cap 18 preferably provides a fluid-tight seal with the second end 14 of the storage container 10 when secured thereto. The storage container 10 with the cap 18 secured thereto, defines an interior volume 10V within the storage container 10.

Optionally, the threaded cap 18 may include a hang tab 20 having a hole 22 therethrough to facilitate hanging the product for sale in a retail establishment, if desired.

Preferably, a plurality of reeds 30 are placed in the storage container 10. Each reed is preferably elongate and slender having a first end 32 and a second end 34. The reeds 30 are preferably made of a porous, wicking material, as for example wood rattan. Each reed has a length 30L defined by the distance between the first and second ends 32 and 34, respectively. Preferably, the reeds 30 forming the plurality of reeds all have approximately the same length. Common reed lengths range from 6″ to 15″, although it is to be understood that the present invention is not limited to these lengths. The reed length 30L is smaller than the storage container length 10L in the preferred embodiments.

Still referring to FIG. 1, the plurality of reeds 30 and a volume of fragranced liquid 40 are placed within the interior volume 10V of the storage container 10. As shown in FIG. 1, the reeds 30 are preferably substantially immersed in the fragranced liquid 40 within the storage container 10. The cap 18 is then sealingly engaged to the second end 14 of the storage container 10 to define a sealed interior chamber within the storage container 10. Once sealed, the packaged assembly of the plurality of reeds 30, the volume of fragranced liquid 40 and the sealed storage container 10 are packaged and ready to ship or distribute to retail outlets, distributors, and end users.

It is to be understood that the plurality of reeds 30 commence drawing, absorbing, wicking and/or becoming saturated with the fragranced liquid 40 upon being packaged. Referring to FIG. 1, even if the fragranced liquid level 40L is below the upper portion of the reeds 30, the hygroscopic nature or the porous, wicking nature of the reeds 30 will draw up the fragranced liquid 40 to the second end 34 of the reeds 30. Preferably, the fragranced liquid level is higher than 50% of the reed length 30L when the storage container 30 is vertically positioned as shown in FIG. 1, and more preferably higher than 75% of the reed length 30L.

It is to be understood that the enlarged diameter at the first end 12 of the storage container 10 in FIG. 1 is provided to facilitate stability of the packaged storage container 10 when placed in an upright position on its base member 26. Alternatively, the packaged storage container 10 could be hung from a rod extending through the hang tab hole 22.

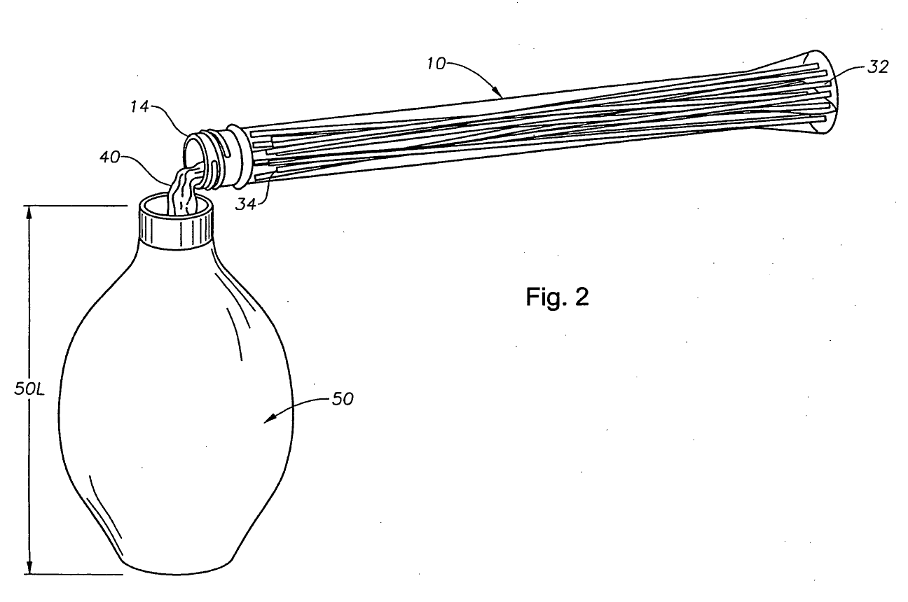

Referring now to FIG. 2, the use of the present invention will now be described. In FIG. 2, the sealed cap 18 has been removed from the storage container 10 and the fragranced liquid 40 is poured from the second end 14 of the storage container 10 into a holding container 50. The plurality of reeds 30 are also transferred from the storage container 10 to the holding container 50. Preferably, the second end 34 of the reeds 30 are placed in the holding container 50. Preferably, the transfer of the storage container contents is accomplished without physically touching the fragranced liquid 40 and the plurality of reeds 30.

The holding container 50 may have a variety of shapes and sizes as is understood by a person of ordinary skill in the art. Preferably, the holding container 50 has a length 50L that is less than the reed length 30L so that when the reeds 30 are placed within the holding container 50 a portion of the reeds 30 extend above the holding container opening 52 as shown in FIG. 3. With the contents of the storage container 10 transferred to the holding container 50, one end of the reeds 30 are supported in the holding container 50 and the other end of the reeds 30 extend upwardly through the opening 52 of the holding container 50. The empty storage container 10 may then be discarded, or alternatively, retained for subsequently returning the reeds 30 and remaining fragranced liquid to the storage container 10 for re-use at a later date.

It is to be understood that upon the transfer to the holding container 50, the presoaked and saturated reeds immediately diffuse fragrance into a room or the surrounding area for ultimate fragrance enjoyment—without any lengthy delay. It is also to be understood that the instant fragrance diffusion is enhanced by the fragranced liquid 40 present on the exposed exterior surface of the reeds 30 outside of the holding container 50.

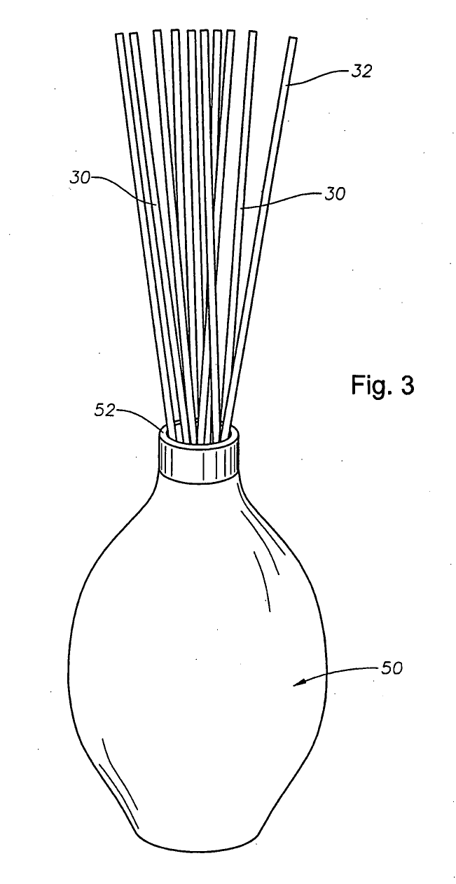

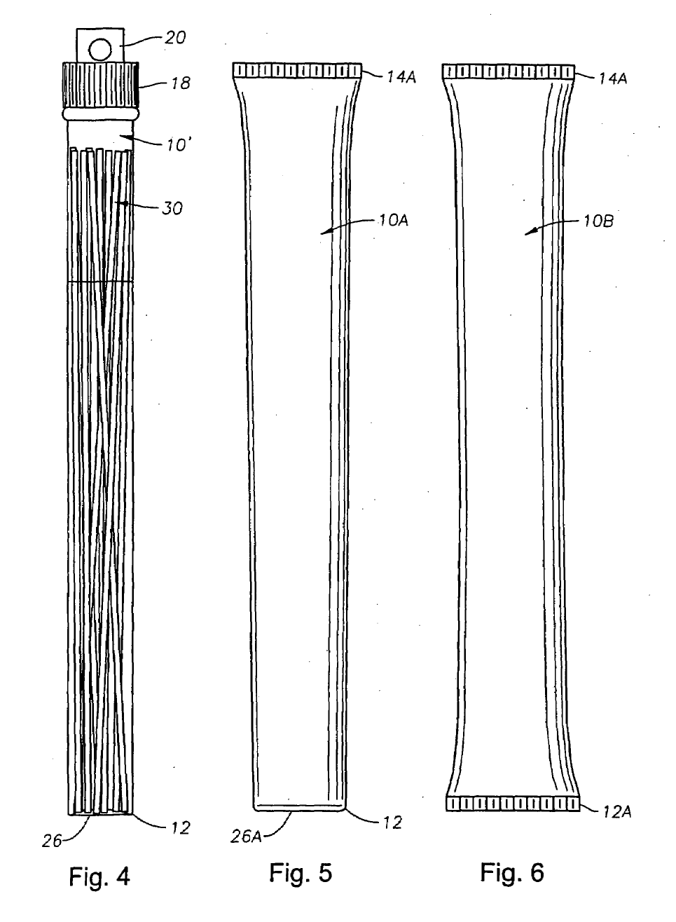

It is to be understood that various constructions and shapes of the storage container 10 may be used in the present invention. An alternative embodiment of the present invention in the packaged condition is shown in FIG. 4. This embodiment is similar to the embodiment described above with the exception that the diameter remains substantially uniform along the entire length of the storage container 10′. This design optimizes space requirements during both shipping and display for sale. It is to be understood that all of the other features are the same as described above.

FIGS. 5 and 6 show alternative embodiments of the storage container according to the present invention, designated 10A and 10B, respectively. The storage container 10A is shown having a substantially circular base 26A at one end 12 and a sealed, flattened distal end 14A. The storage container 10A preferably being made of a fluid-tight, sturdy plastic material. As described above, the plurality of reeds 30 (not shown) and the fragranced liquid 40 (not shown) are placed in the storage container 10A prior to sealing the flattened end 14A. Preferably, the flattened end 14A is cut off, as for example with a scissors, to transfer the contents to the holding container 50. The storage container 10B is similar to the storage container 10A with the exception that the substantially circular base 26A of FIG. 5 has been replaced with a flattened end 12A. Thus, the ends 12A and 14A of the storage container 10B are sealed, flattened ends. Although not shown in FIGS. 5 and 6, it is to be understood that the storage containers 10A and 10B could additionally include a hang tab 20 with hole 22 as shown in FIG. 1. Preferably, the hang tab 20 would be attached to an end of the storage container 10A, 10B.

It is to be understood that the storage containers according to the preferred embodiment of the present invention may be transparent to allow the contents to be seen without opening the storage container. However, it is also to be understood that the storage containers do not need to be transparent.

It is to be understood that the storage container according to the present invention is not limited to the illustrated designs. According to the preferred embodiment of the present invention, the storage container includes an interior chamber sized to receive the plurality of reeds and the fragranced liquid. The storage container can be arranged and designed to hang, stand upright, be positioned horizontally or in various other positions. The storage container may have a cross-section that is generally circular, elliptical, flattened or polygonal.

Preferably, at least one end of the reeds are in the fragranced liquid when in the storage container. This allows the reeds to wick or absorb the fragranced liquid throughout the length of the porous, wicking material of the reeds while in the storage container. Thus, once the reeds are removed from the storage container and placed in the holding container, the fragranced liquid is at the exposed end of the reeds to immediately commence diffusing the fragrance.

Preferably, a majority of the outer surface area of the reeds is in contact with the fragranced liquid when in the storage container, and more preferably substantially all the outer surface area of the reeds is in contact with the fragranced liquid. This allows the fragranced liquid to coat and partially soak into the exterior surface of the reeds. Thus, once the reeds are removed from the storage container and placed in the holding container, the exposed coated portions of the reeds extending from the mouth of the holding container immediately commence releasing the fragrance.

Thus, it is to be understood that the present invention provides immediate fragrance to a room using reeds and fragranced liquid without any lengthy delay.

A study was conducted to determine whether a difference exists in the scent intensity between the pre-soaked fragrant reeds according to the present invention as compared to prior art dry non-soaked reeds at two separate time intervals—“just opened” and “after 24 hours elapsed time.” A consumer sample of 25 people (12 male and 13 female) compared the scent intensity of “just opened/immediate” and “after 24 hours elapsed time” pre-soaked fragrant reeds with the control product of dry, non-soaked reeds as currently available in the market. A sub-sample of 19 people (6 male and 13 female) of the sample 25 people compared the two products after being opened for 24 hours. Both tested products were identical in the following respects: same fragrance of Island Orchard, same formula of 10% load, same fragrance level, same vessel (3½″ tall vase having a vase opening varying from ¾″ to 1¼″ diameter), same amount of fragranced liquid (6 ounces), and same number and size of reeds (eight (8) 10″ long rattan reeds). The pre-soaked reeds had soaked in the fragranced liquid for approximately two weeks prior to the study.

The pre-soaked product was displayed in a vase with the pre-soaked rattan reeds inserted and the remaining fragranced liquid poured in the vase. The control product was displayed with the fragranced solution poured into the vase and the dry reeds immediately inserted into the vase. The products were placed in separate rooms of the same size (approximately 20′ by 30′) at the same location.

The consumer sample, divided into groups of 10-12, were brought into each room and the consumers were asked to rate the intensity of the fragrance and their preference: How would you rate the intensity of the fragrance of these products? (measured on a 5-point scale where 1 is poor and 5 is excellent); and which of the two products do you prefer? The consumer sample was informed as to how the products differed. The tables below show the detailed results of the study, i.e., the ratings of the two products when “just opened” and “after 24 hours.” The sample sizes vary due to six people not participating in the second phase (after 24 hours).

| | |
|---|---|
| Just Opened | After 24 Hours |

| Pre-soaked | | Pre-soaked | |
|---|---|---|---|
| Reeds | Dry Reeds | Reeds | Dry Reeds |
| 4 | 1 | 5 | 2 |
| 5 | 1 | 5 | 2 |
| 5 | 1 | 4 | 2 |
| 5 | 1 | 4 | 2 |
| 5 | 1 | 4 | 1 |
| 4 | 2 | 3 | 1 |
| 4 | 2 | 4 | 2 |
| 5 | 1 | 4 | 2 |
| 5 | 1 | 5 | 2 |
| 5 | 1 | 3 | 1 |
| 4 | 1 | 4 | 2 |
| 4 | 3 | 4 | 1 |
| 4 | 1 | 5 | 1 |
| 4 | 2 | 5 | 2 |
| 5 | 1 | 3 | 2 |
| 4 | 1 | 4 | 2 |
| 5 | 2 | 4 | 2 |
| 4 | 1 | 3 | 2 |
| 4 | 1 | 3 | 2 |
| 4 | 2 | | |
| 4 | 1 | | |
| 4 | 1 | | |
| 4 | 2 | | |
| 4 | 2 | | |
| 4 | 1 | | |
| 4.36 | 1.36 | 4.0 | 1.74 |

5-Point Scale (1: poor, 2: fair, 3: good, 4: very good, 5: excellent)

The sample of consumers overwhelmingly rated the pre-soaked fragrant reeds superior in scent intensity to the dry reeds in both the “just opened/immediate” and “after 24 hours elapsed time” intervals. In the “just opened” test, the average scores for the pre-soaked fragrant reeds across the 25 respondents was 4.36 compared to 1.36 for the dry product. In the “after 24 hours” test, the average scores for the pre-soaked fragrant reeds across the 19 respondents was 4.0 compared to 1.74 for the dry product. Not only are these differences significant, but every person (100% of respondents) ranked the pre-soaked fragrant reeds superior to dry reeds and for both elapsed time intervals (immediate and after 24 hours). When asked which product they preferred, 76% (19 of 25) preferred the pre-soaked fragrant reeds over the dry reeds when it was immediately opened, and 79% (15 of 19) preferred the pre-soaked fragrant reeds over the dry reeds after 24 hours. The study clearly demonstrates the superior “instant fragrance” impact of the pre-soaked fragrant reeds compared to dry reeds when the product is just opened, as well as the superior scent intensity impact after an elapsed time of 24 hours. Furthermore, the data clearly indicates a consumer preference for the pre-soaked reeds over the dry reeds.

As previously stated, various commercially available, prior art fragrant reed products indicate that, for reeds having a length of approximately 10″, the reeds will draw and diffuse the fragrance within approximately 36 to 48 hours. The present invention eliminates the lengthy wait and immediately and continuously releases and diffuses the fragrance throughout the surroundings upon placement in the holding container.

The foregoing disclosure and description of the invention are illustrative and explanatory thereof, and various changes in the size, shape and materials, as well as the details of the illustrated operation and construction may be made without departing from the spirit and scope of the invention.

I claim:

1. A packaged kit for storing an instant fragrance dispensing assembly comprising:
a storage container having a sealed interior chamber;
a plurality of reeds within said sealed interior chamber; and
a fragranced liquid within said sealed interior chamber, said fragranced liquid contacting at least one of said plurality of reeds.

2. The packaged kit of claim 1, wherein said fragranced liquid contacts each of said plurality of reeds in said sealed interior chamber.

3. The packaged kit of claim 1, wherein said reeds are elongate.

4. The packaged kit of claim 3, wherein said reeds are made of a porous, wicking material.

5. The packaged kit of claim 4, wherein said fragranced liquid contacts a porous end of each of said plurality of reeds in said sealed interior chamber.

6. The packaged kit of claim 2, wherein said plurality of reeds have an outer surface area, and a majority of said outer surface area of said plurality of reeds is in contact with said fragranced liquid when in said storage container.

7. The packaged kit of claim 2, wherein said plurality of reeds have an outer surface area, and substantially all of said outer surface area of said plurality of reeds is in contact with said fragranced liquid when in said storage container.

8. The packaged kit of claim 1, wherein said storage container is elongate and has first and second ends, and a hang tab is attached to one of said ends.

9. The packaged kit of claim 2, wherein each of said reeds are elongate and have a reed length and said storage container is elongate and has a lower end when said elongate storage container is vertically oriented, and said fragranced liquid has a liquid level above said lower end, when said elongate storage container is vertically oriented, that is greater than 50% of said reed length.

10. The packaged kit of claim 2, wherein each of said reeds are elongate and have a reed length and said storage container is elongate and has a lower end when said elongate storage container is vertically oriented, and said fragranced liquid has a liquid level above said lower end, when said elongate storage container is vertically oriented, that is greater than 75% of said reed length.

11. The packaged kit of claim 2, wherein said storage container includes a replaceable cap.

12. The packaged kit of claim 1, further comprising a holding container arranged and designed to receive and contain said fragranced liquid and said plurality of reeds.

13. A method of packaging a plurality of reeds and a volume of fragranced liquid, the method comprising the steps of:
inserting the volume of fragranced liquid in a storage container;
inserting the plurality of reeds in the storage container;
placing the plurality of reeds in contact with the volume of fragranced liquid; and
sealing the storage container.

14. The method of claim 13, wherein the plurality of reeds have an outer surface area, and a majority of the outer surface area of the plurality of reeds is in contact with the fragranced liquid when in the storage container.

15. A method of providing instant and continuous fragrance to a room using a plurality of reeds and a fragranced liquid, the method comprising the steps of:
opening a sealed storage container containing the plurality of reeds in the fragranced liquid;
transferring the plurality of reeds and the fragranced liquid from the storage container to a holding container; and
allowing a portion of the plurality of reeds to extend from the holding container.

16. The method of claim 15, wherein the holding container has an opening remaining open.

17. The method of claim 15, wherein the plurality of reeds have an outer surface area, and a majority of the outer surface area of the plurality of reeds is in contact with the fragranced liquid when in the sealed storage container.

18. The method of claim 15, where said step of transferring the plurality of reeds and the fragranced liquid comprises pouring the contents from the storage container into the holding container.
